(19) United States
(12) Patent Application Publication
(10) Pub. No.: US 2006/0287500 A1
(43) Pub. Date: Dec. 21, 2006

(54) Method for synthesis of C2-symmetric diamino diol mediated by titanium complexes

(75) Inventors: Chun-Chih Wang, Yilan County (TW); Shyh-Fong Chen, Taipei City (TW); Shan-Yen Chou, Taipei City (TW); Kae-Shyang Shih, Taipei City (TW); Shieh-Shung T. Chen, Taipei City (TW)

Correspondence Address: BIRCH STEWART KOLASCH & BIRCH, PO BOX 747, FALLS CHURCH, VA, 22040-0747, US

(73) Assignee: Development Center for Biotechnology

(21) Appl. No.: 11/253,152

(22) Filed: Oct. 19, 2005

(30) Foreign Application Priority Data
Jun. 21, 2005 (TW) 94120570

(51) Int. Cl. C07C 209/50; C07K 7/06

(52) U.S. Cl. 530/329; 564/372

(57) ABSTRACT

A method for synthesis of C₂-symmetric diamino diol mediated by titanium complexes is provided. A substituted-L-phenylalaninal undergoes pinacol coupling to yield the corresponding C₂-symmetric (1S,2R,3R,4S)-1,4-diamino 2,3-diol in the presence of Cp₂TiCl₂/ZnCl₂ and zinc metal, mediated in good yield and highly selective. This titanium-catalyzed reaction yields diaminodiol, offering a convenient alternative method to the synthesis of C₂-symmetric peptidic protease inhibitors. Consequently, the method allows to synthesize TL-3 via titanium complex in moderate yield.

### BACKGROUND

The invention relates to a method for synthesis of C₂-symmetric diamino diols, especially C₂-symmetric (1S,2R,3R,4S)-1,4-diamino 2,3-diol, mediated by titanium complexes.

Human immunodeficiency virus (HIV) has been shown to be the causative agent of acquired immunodeficiency syndrome (AIDS). On the basis of X-ray crystal data which shows that HIV protease exists as a C₂-symmetric dimmer, a number of C₂-symmetric peptidic and non-peptidic HIV protease inhibitors have been investigated. The active sites of HIV proteases are C₂-symmetric. Therefore, a series of novel inhibitors having a C₂-symmetry designed to co-align with the C₂-axis of the enzymes are investigated (WO99/29311; U.S. Pat. No. 5,362,912).

C₂-symmetric (1S,2R,3R,4S)-1,4-diamino 2,3-diol is useful for synthesizing important intermediates for C₂-symmetric peptidic HIV protease inhibitors. The traditional methods for synthesizing (1S,2R,3R,4S)-1,4-diamino 2,3-diol derivatives are catalyzed by [V₂Cl₃(THF)₆]₂[Zn₂Cl₆], using Pedersen's procedure. Treatment of L-(N-benzyloxycarbonyl)-phenylalaninal in the presence of [V₂Cl₃(THF)₆]₂[Zn₂Cl₆] and zinc metal powder in CH₂Cl₂ at room temperature for 16 h led to homocoupling give diaminodiol in 76% yield, provide a mixture with a ratio of 80/10/10 of (1S,2R,3R,4S)/(1S,2S,3S,4S)/(1S,2R,3S,4S), respectively (J. Org. Chem. 1992, 57, 28).

One inhibitor of TL-3 is C₂-symmetric diol, effectively inhibited FIV protease and HIV protease in vitro (Kᵢ of 41 and 1.5 nM, respectively). In feline model, TL-3 demonstrated a superior profile against drug-resistance. No evidence for drug resistance developing against TL-3 was observed. Additional studies showed that TL-3 was equally effective against HIV, SIV, FIV and many of the 150 different clinical mutant strains (J. Am. Chem. Soc. 1999, 121, 1145).

However, the traditional methods for synthesizing TL3 protease inhibitor derivatives are limited by poor overall yield (5.3%) and harsh reaction conditions (11 steps). In addition, H₂ gas was used.

### SUMMARY

The invention provides a method for synthesis of C₂-symmetric diamino diol.

In one aspect, the present invention features a method for synthesis of C₂-symmetric diamino diol via a titanium-catalyzed pinacol coupling.

In another aspect, the present invention features a method for synthesis of TL-3 protease inhibitor via a titanium-catalyzed pinacol coupling.

Other advantages or features of this invention will be apparent from the following detailed description thereof.

### DETAILED DESCRIPTION

An object of the invention is a method for synthesis of C₂-symmetric diamino diol, comprising pinacol coupling substituted L-phenyl alaninal in the present of Ti catalyst,

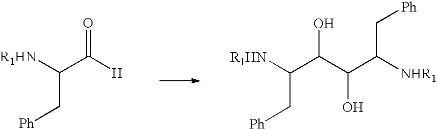

wherein R₁ is selected from at least one of the group consisting of

benzyloxycarbonyl-, t-butyloxycarbonyl-, carbobenzyloxy-valine-, carbobenzyloxy-alanine-, carbobenzyloxy-leucine-, carbobenzyloxy-serine-, carbobenzyloxy-glycine-, carbobenzyloxy-threonine-, carbobenzyloxy-asparagine-, t-butyloxycarbony-valine-, t-butyloxycarbony-alanine-, t-butyloxycarbony-leucine-, t-butyloxycarbony-serine-, t-butyloxycarbony-glycine-, t-butyloxycarbony-threonine-, t-butyloxycarbony-asparagine-, carbobenzyloxy-alanine-valine-, carbobenzyloxy-alanine-asparagine-, carbobenzyloxy-serine-valine-, carbobenzyloxy-valine-valine-, carbobenzyloxy-leucine-valine-, carbobenzyloxy-phenylalanine-valine-, carbobenzyloxy-glycine-valine-, t-butyloxycarbony-serine-valine-, t-butyloxycarbony-threonine-valine-, t-butyloxycarbony-asparagine-alanine and t-butyloxycarbony-asparagine-valine.

The Ti catalyst comprises:

TiX₄, TiX₃, TiX₃L, TiX₂L₂ or TiL₄;

wherein X is selected from the group consisting of Cl, Br and I; and

L is selected from the group consisting of cyclopentadienyl, tetrahydrofuran, t-butylcyclopentadienyl, ethylcyclopentadienyl and i-propylcyclopentadienyl.

The reaction can further comprise Zn and/or ZnCl₂ as a catalyst.

The reaction can be performed under 20-30° C., preferably 25° C.

The reaction can be performed 8 to 24 hours, preferably 12 hours.

In this reaction, the substituted L-phenyl alaninal is dissolved in THF before pinacol coupling.

The C₂-symmetric diamino diol can be a C₂-symmetric (1S,2R,3S,4S)-1,4-diamino-2,3-diol,

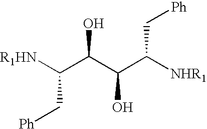

Alternatively, the C₂-symmetric diamino diol can be a TL-3 protease inhibitor as the compound shown as

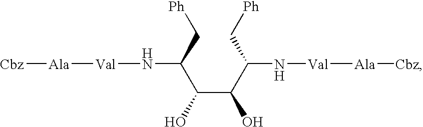

which is known efficiently against HIV, FIV or SIV.

In the followings, an embodiment of the method for synthesis of C₂-symmetric diamino diol is illustrated in Scheme 1, and another embodiment of the method for synthesis of TL-3 protease inhibitor is shown in Scheme 2. The abbreviations in the scheme 1 and 2 represent as:
BAIB Iodobenzene diacetate
Cbz Benzyloxycarbonyl
Cbz-Cl Benzyloxycarbonylchloride
Cp₂TiCl₂ Biscyclopentane titanium dichloride
DCM Dichloromethane
EDC (1-(3-dimethyaminopropyl)-3-ethylcarbodiimide hydrochloride
HOBt N-hydroxybenzotriazole
LAH Lithium aluminum hydride
NEt₃ Trimethylamine
Ph Phenyl
TEMPO, 2,2,6,6-tetramethyl-1-piperidinyloxy, free
free radical
radical
THF Tetrahydrofuran

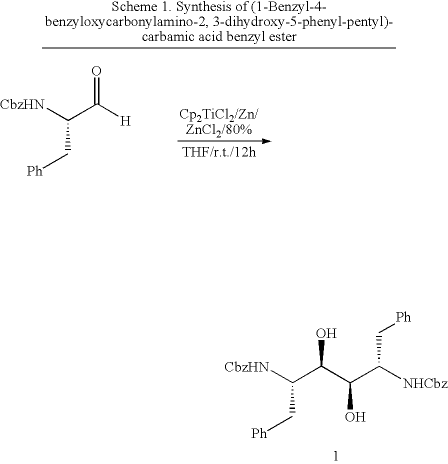

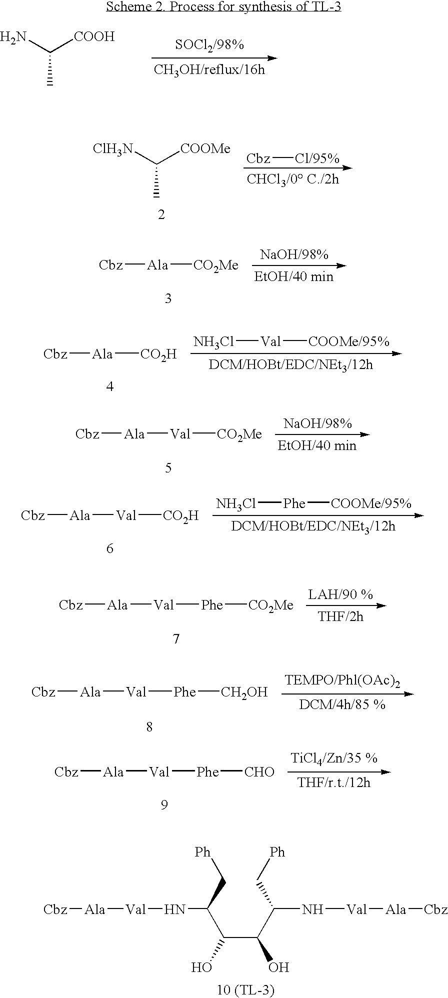

The method for synthesis of C₂-symmetric diamino diol via a titanium-catalyzed pinacol coupling has 80% yield (Scheme 1). In addition, the reaction is highly stereoselective and provide a crude with a ratio of 85/10/5 mixture of (1S,2R,3R,4S)/(1S,2S,3S,4S)/(1S,2R,3S,4S), respectively. The reaction is more effective than the conventional Pedersen's procedure.

The titanium-catalyzed pinacol coupling mode of the present invention also provides several advantages: (a) Cp₂TiCl₂ (100 g/US$=125) is cheaper than [V₂Cl₃(THF)₆] (25 g/US$=105); (b) The catalyst of Cp₂TiCl₂ is not moisture sensitive compare to vanadium complex so that the present titanium-catalyzed reaction provides a very convenient method for the one-pot synthesis of (1S,2R,3R,4S)-1,4-diamino 2,3-diol under mild conditions; and (c) L-(N-benzyloxycarbonyl)-phenylalaninal and another amino aldehyde were well dissolved in THF caused to easily react.

The TL-3 protease inhibitor produced by the method for synthesis of C₂ symmetric diamino diol has 24% overall yield (Scheme 2), thereby improving conventional low-yield, stringent conditions and no H₂ gas is used.

Other characteristics and advantages of the invention will become apparent in the continuation of the description with the examples represented below. In theses examples, reference will be made to Scheme 1 and Scheme 2. The examples have the purpose to illustrate the invention and are not to be considered as limitation of the same.

### EXAMPLE 1

### Synthesis of (1-Benzyl-4-benzyloxycarbonylamino-2,3-dihydroxy-5-phenyl-pentyl)-carbamic acid benzyl ester

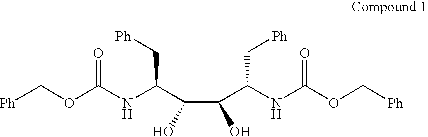

### (1-Benzyl-4-benzyloxycarbonylamino-2,3-dihydroxy-5-phenyl-pentyl)-carbamic acid benzyl ester

To a 25-mL sidearm flask were added Cp₂TiCl₂ (0.266 g, 1 mmol,), zinc metal powder (3.0 mmol), ZnCl₂ (0.5 mmol) and L-(N-benzyloxycarbonyl)-phenylalaninal (0.286 g, 1.00 mmol). The system was evacuated and purged with nitrogen three times. Freshly distilled dry THF (3.0 mL), were added to the flask via syringes. The reaction mixture was stirring at room temperature for 4 h, and diluted with dichloromethane and water, stirred in the air for 10 min. The mixture was filtered through a Celite and silica gel pad and washed with dichloromethane. The phases were separated, and the organic layer was washed by saturated brine. Dried (magnesium sulfate) gave after filtration and concentration 0.22 g (80%) of the desired product as white solid. ¹H NMR (500 MHz, CDCl₃): δ 9.55 (s, 2H), 7.30-7.05 (m, 20H), 5.30 (br, 2H), 5.03 (s, 4H), 4.43 (d, J=6.5 Hz, 2H), 3.06 (d, J=6.5 Hz, 4H).

### EXAMPLE 2

### Synthesis of TL-3 (Scheme 2)

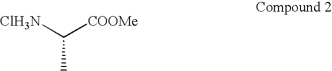

A 5.34 g (60 mmol) sample of L-alanine was slurried in 100 ml of CH₃OH. To this was slowly added 6 ml of thionyl chloride, the temperature was allowed to increase, and the solution held at reflux for 16 hours. The solvent was removed to afford the desired product) as a white solid. The compound was taken directly to the next step. ¹H NMR (500 MHz, CDCl₃): δ 8.63 (s, 3H), 4.28 (q, J=6.5 Hz, 1H), 3.78 (s, 3H), 1.70 (d, J=6.5 Hz, 3H). ¹³C NMR (125 MHz, CDCl₃): δ 170.71, 53.33, 49.40, 16.07.

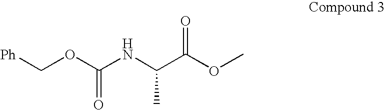

The crude compound 2 (3.378 g, 24.2 mmol) was dissolved in 40 ml of chloroform and cooled to 0° C. To this was added 40 ml of 20% sodium carbonate, and stirred for an additional 15 min to complete the neutralization. The solution was cooled to 0° C., 8 ml (27.8 mmol) of 50% benzyl chloroformate was added slowly, and the reaction stirred for 2 h at room temperature. The layers were separated, and the chloroform layer was washed with 20 ml of 1N hydrochloric acid and dried over magnesium sulfate. The solvent was removed to give 5.45 g (95%) of the desired product as clear oil. ¹H NMR (500 MHz, CDCl₃): δ 7.36-7.30 (m, 5H), 5.34 (br, 1H), 5.11 (s, 2H), 4.39 (q, J=7.0 Hz, 1H), 3.74 (s, 3H), 1.41 (d, J=7.0 Hz, 3H). ¹³C NMR (125 MHz, CDCl₃): δ 173.61, 155.70, 136.34, 128.66, 128.31, 128.25, 67.05, 52.61, 49.69, 18.82.

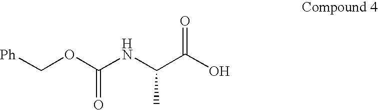

5 g (21 mmol) of compound 3 was dissolved in 50 ml of ethanol, 30 ml of 2.5 mM NaOH was added. The reaction was stirred at room temperature for 40 minutes, acidified with 6N HCl to about pH 2. The combined extracts were washed twice with saturated NaCl solution, dried (Na₂SO₄). The solvent was removed to give 4.59 g (98%) of the desired product as white solid or clear oil. ¹H NMR (500 MHz, CDCl₃): δ 7.34-7.27 (m, 5H), 5.39 (br, 1H), 5.11 (s, 2H), 4.38 (q, J=7.2 Hz, 1H), 1.43 (d, J=7.2 Hz, 3H). ¹³C NMR (125 MHz, CDCl₃): δ 177.78, 155.98, 136.16, 128.68, 128.39, 128.28, 67.27, 49.59, 18.49.

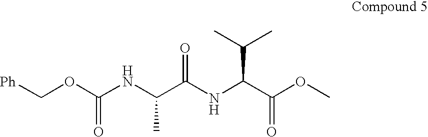

5 g (22.4 mmol) of compound 4 and 3.7 g (22.4 mmol) L-valine methyl ester hydrochloride were dissolved in 120 ml of CH₂Cl₂ and cooled to 0° C. HOBt (4.54 g, 33.6 mmol) and NEt₃ (0.93 ml, 67.2 mmol) were added, followed by EDC (5.37 g, 28 mmol) after 15 min. After additional 15 min at 0° C. the mixture was stirred at the room temperature for 12 h. The combined organic layers were washed with saturated NaCl solution, dried with Na₂SO₄. The solvent was removed to give 7.15 g (95%) of the desired product as white solid. ¹H NMR (500 MHz, CDCl₃): δ 7.35-7.30 (m, 5H), 6.53 (br, 1H), 5.34 (br, 1H), 5.11 (s, 2H), 4.52 (q, J=7.0 Hz, 1H), 4.27 (br, 1H), 3.73 (s, 3H), 2.16-2.14 (m, 1H) 1.38 (d, J=7.0 Hz, 3H), 0.90-0.86 (m, 6H). ¹³C NMR (125 MHz, CDCl₃): δ 172.33, 172.25, 156.11, 136.27, 128.68, 128.35, 128.22, 67.19, 57.24, 52.36, 50.61, 31.34, 19.02, 18.37, 17.76.

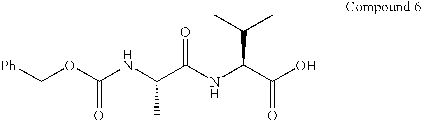

7.05 g (21 mmol) of compound 5 was dissolved in 50 ml of ethanol, 30 ml of 2.5 mM NaOH was added. The reaction was stirred at room temperature for 40 minutes, acidified with 6N HCl to about pH 2. The combined extracts were washed twice with saturated NaCl solution, dried (Na₂SO₄). The solvent was removed to give 4.59 g (98%) of the desired product as white solid or clear oil. ¹H NMR (500 MHz, CDCl₃): δ 9.45 (br, 1H), 7.33-7.27 (m, 5H), 7.05 (d, J=8.5 Hz, 1H), 5.91 (d, J=8.5 Hz, 1H), 5.07 (s, 2H), 4.52 (q, J=7.2 Hz, 1H), 4.38-4.35 (m, 1H), 2.20-2.17 (m, 1H), 1.33 (d, J=7.0 Hz, 3H), 0.9-0.86 (m, 6H). ¹³C NMR (125 MHz, CDCl₃): δ 174.71, 173.46, 156.38, 136.17, 128.60, 128.28, 128.09, 67.19, 57.31, 50.55, 31.06, 21.16, 19.01, 17.59.

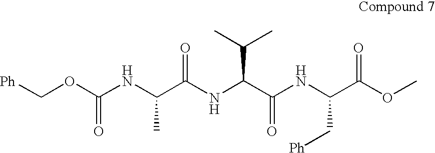

8.0 g (24.2 mmol) of compound 6 and 5.2 g (24.2 mmol) of L-phenylalanine methyl ester hydrochloride were dissolved in 140 ml of CH₂Cl₂ and cooled to 0° C. HOBt (4.9 g, 36.3 mmol) and NEt₃ (10 ml, 72.6 mmol) were added, followed by EDC (5.8 g, 30.2 mmol) after 15 min. After additional 15 min at 0° C. the mixture was stirred at the room temperature for 12 h. The combined organic layers were washed with saturated NaCl solution, dried with Na₂SO₄. The solid residue was triturated with a small amount of CH₂Cl₂ (to remove some yellow impuries), give 11.1 g (95%) of the desired product as white solid. ¹H NMR (500 MHz, CDCl₃): δ 7.35-7.31 (m, 4H), 7.28-7.22 (m, 4H), 7.09-7.07 (m, 2H), 6.62 (br, 1H), 6.40 (br, 1H), 5.38 (br, 1H), 5.09 (s, 2H), 4.85 (q, J=7.0 Hz, 1H), 4.25-4.20 (m, 2H), 3.71 (s, 3H), 3.12-3.04 (m, 2H), 2.08 (br, 1H) 1.35 (d, J=7.0 Hz, 3H), 0.89-0.83 (m, 6H). ¹³C NMR (125 MHz, CDCl₃): δ 172.34, 171.76, 170.58, 156.12, 136.22, 135.69, 129.32, 128.79, 128.69, 128.38, 128.26, 127.35, 67.24, 58.53, 53.24, 52.52, 50.74, 37.90, 31.06, 19.16, 18.49, 17.93.

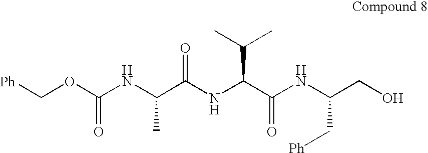

3.41 g (7 mmol) of lithium aluminum hydride was dissolved in 50 ml THF and stirred under N₂ at 0° C. The 6 g (12.4 mmol) of compound 7 was added slowly via powder addition funnel, the mixture was allowed to warm to room temperature and stirred for an additional hour. The reaction was quenched by careful addition of 30 ml of water, 30 ml of 15% NaOH, the resultant gel was filtered, and the filter cake was washed thoroughly with hot ethyl acetate. The solvent was removed to give 8.19 g (90%) of the desired product as white solid. ¹H NMR (500 MHz, CDCl₃): δ 7.34-7.26 (m, 5H), 7.25-7.22 (m, 5H), 6.60-6.55 (br, 2H), 5.29 (br, 1H), 5.12 (s, 2H), 4.18-4.12 (m, 3H), 3.71 (m, 1H), 3.55 (m, 1H), 2.85 (m, 2H), 2.21 (br, 1H) 1.38 (d, J=7.0 Hz, 3H), 0.86(d, J=7.0 Hz, 3H), 0.77 (d, J=7.0 Hz, 3H). ¹³C NMR (125 MHz, CDCl₃): δ172.56, 170.76, 156.49, 137.77, 135.73, 129.19, 128.66, 128.54, 128.47, 128.17, 126.55, 67.47, 63.71, 59.12, 52.88, 51.40, 36.76, 29.71, 19.24, 17.98, 17.43.

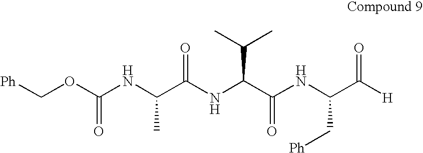

To a solution of the crude alcohol compound 8 (4.6 g, 10 mmol) in DCM (100 ml) was added TEMPO (0.37 g, 2.5 mmol) and BAIB (8 g, 25 mmol), the mixture was refluxed for 2 h. The mixture was diluted with DCM, washed with saturated Na₂S₂O₃, saturated NaHCO₃, brine, dried over MgSO₄ and filter. The solvent was removed and washed with the mixture of Hex/EA (1:1) to give 3.6 g (80%) of the desired product as white solid. ¹H NMR (500 MHz, CDCl₃): δ 9.59 (s, 1H), 7.37-7.25 (m, 8H), 7.14 (d, J=7.0 Hz, 2H), 6.62 (br, 1H), 6.59 (br, 1H), 5.33 (br, 1H), 5.09 (s, 2H), 4.7 (q, J=7.0 Hz, 1H), 4.26-4.21 (m, 2H), 3.14-3.10 (m, 2H), 2.14 (br, 1H), 1.36 (d, J=7.0 Hz, 3H), 0.89 (d, J=7.0 Hz, 3H), 0.83 (d, J=7.0 Hz, 3H). ¹³C NMR (125 MHz, CDCl₃): δ198.69, 172.53, 171.18, 156.29, 136.18, 135.66, 129.34, 128.91, 128.71, 128.41, 128.22, 127.30, 67.28, 59.75, 58.61, 50.83, 35.09, 30.76, 19.28, 18.45, 17.92.

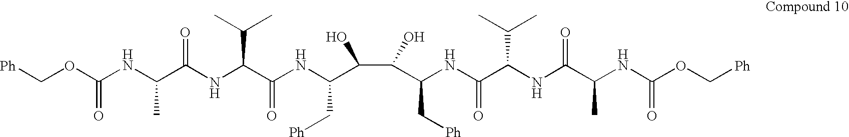

To a 25-mL sidearm flask were added zinc metal powder (4 mmol) and compound 9 (0.453 g, 1.00 mmol). The system was evacuated and purged with nitrogen three times. Freshly distilled dry THF (6.0 mL), was added to the flask via syringes. TiCl₄ (4 mmol) was added dropwise, followed by H₂O (36 mg, 2 mmol) and the reaction mixture was stirring at room temperature for 12 h, diluted with dichloromethane and 15 ml of 6N HCl, stirred in the air for 20 min. The phases were separated, and the organic layer was washed by saturated brine. Dried (magnesium sulfate) gave after filtration and concentration 0.22 g (50%) of the desired product as white solid. The product was isolated and purified as usual in 35% yield. ¹H NMR (500 MHz, DMSO-d₆): δ 7.55-7.09 (m, 26H), 5.02 (s, 4H), 4.47 (br, 2H), 4.07 (m, 4H), 3.25 (br, 2H), 2.77 (m, 2H), 2.60 (m, 2H), 1.80 (m, 2H), 1.16 (s, 3H), 1.15 (s, 3H), 0.71 (d, J=6.5 Hz, 6H), 0.67 (d, J=6.5 Hz, 6H).

### COMPARATIVE EXAMPLE

### Synthesis of (1S,2R,3R,4S)-1,4-diamino 2,3-diol catalyzed by [V₂Cl₃(THF)₆]₂[Zn₂Cl₆], using Pedersen's procedure

L-(N-benzyloxycarbonyl)-phenylalaninal in the presence of [V₂Cl₃(THF)₆]₂[Zn₂Cl₆] and zinc metal powder in CH₂Cl₂ at room temperature for 16 h led to homocoupling give diaminodiol in 35% yield, provided a mixture of a ratio 87/8/5 of (1S,2R,3R,4S)/(1S,2S,3S,4S)/(1S,2R,3S,4S), respectively.

### OTHER EMBODIMENTS

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Therefore, the scope of the appended claims should be accorded the broadest interpretation to encompass all such modifications and similar arrangements.

What is claimed is:

1. A method for synthesis of C₂-symmetric diamino diol, comprising pinacol coupling a substituted L-phenyl alaninal in a reaction system comprising a Ti catalyst, 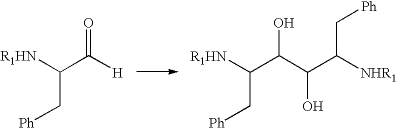
wherein R₁ is selected from the group consisting of
benzyloxycarbonyl-, t-butyloxycarbonyl-, carbobenzyloxy-valine-, carbobenzyloxy-alanine-, carbobenzyloxy-leucine-, carbobenzyloxy-serine-, carbobenzyloxy-glycine-, carbobenzyloxy-threonine-, carbobenzyloxy-asparagine-, t-butyloxycarbony-valine-, t-butyloxycarbony-alanine-, t-butyloxycarbony-leucine-, t-butyloxycarbony-serine-, t-butyloxycarbony-glycine-, t-butyloxycarbony-threonine-, t-butyloxycarbony-asparagine-, carbobenzyloxy-alanine-valine-, carbobenzyloxy-alanine-asparagine-, carbobenzyloxy-serine-valine-, carbobenzyloxy-valine-valine-, carbobenzyloxy-leucine-valine-, carbobenzyloxy-phenylalanine-valine-, carbobenzyloxy-glycine-valine-, t-butyloxycarbony-serine-valine-, t-butyloxycarbony-threonine-valine-, t-butyloxycarbony-asparagine-alanine and t-butyloxycarbony-asparagine-valine.

2. A method for synthesis of C₂-symmetric diamino diol as claimed in claim 1, wherein the Ti catalyst comprises TiX₄, TiX₃, TiX₃L, TiX₂L₂ or TiL₄; in which X is Cl, Br, or I, and
L is selected from the group consisting of cyclopentdienyl, tetrahydrofuran, t-butylcyclopentadienyl, ethylcyclopentadienyl and i-propylcyclopentadienyl.

3. A method for synthesis of C₂-symmetric diamino diol as claimed in claim 1, wherein the reaction system comprises Zn.

4. A method for synthesis of C₂-symmetric diamino diol as claimed in claim 1, wherein the reaction system comprises ZnCl₂.

5. A method for synthesis of C₂-symmetric diamino diol as claimed in claim 1, wherein the reaction system is controlled at 20-30° C.

6. A method for synthesis of C₂-symmetric diamino diol as claimed in claim 1, wherein the pinacol coupling is performed 8 to 24 hours.

7. A method for synthesis of C₂-symmetric diamino diol as claimed in claim 1, wherein the substituted L-phenyl alanine is dissolved in tetrahydrofuran before the pinacol coupling.

8. A method for synthesis of C₂-symmetric diamino diol as claimed in claim 1, wherein the C₂-symmetric diamino diol is C₂-symmetric (1S,2R,3S,4S)-1,4-diamino-2,3-diol

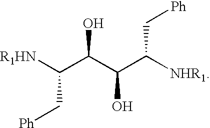

9. A method for synthesis of C₂-symmetric diamino diol as claimed in claim 1, wherein the C₂-symmetric diamino diol is a TL-3 protease inhibitor as the compound

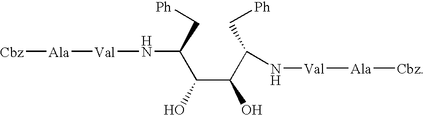

10. A method for synthesis of C₂-symmetric diamino diol as claimed in claim 9, wherein the TL-3 protease inhibitor is against HIV, FIV or SIV.
